(19) United States
(12) Patent Application Publication
(10) Pub. No.: US 2006/0165795 A1
(43) Pub. Date: Jul. 27, 2006

(54) Medicinal compositions comprising a core and a film based on modified cellulose derivatives

(76) Inventor: Kirsty Sawicka, Nottingham (GB)

Correspondence Address: JENKINS, WILSON, TAYLOR & HUNT, P. A., 3100 TOWER BLVD, SUITE 1200, DURHAM, NC, 27707, US

(21) Appl. No.: 10/539,094

(22) PCT Filed: Dec. 15, 2003
(86) PCT No.: PCT/GB03/05472

§ 371 Date: Dec. 19, 2005

(30) Foreign Application Priority Data
Dec. 16, 2002 (GB) 0229258.9

(51) Int. Cl. A61K 31/717; A61K 9/24

(52) U.S. Cl. 424/472; 514/57

(57) ABSTRACT

A medicinal composition comprising: (a) a core in combination with a medicinally effective unit dose of one or more active medicaments; (b) the core being enclosed within a non-expanded film material comprising a modified cellulose material.

The present invention relates to medicinal compositions, in particular although not exclusively, medicinal compositions which are easier to administer to patients such as children, the aged or the infirm who have difficulty swallowing solid dosage forms such as tablets and capsules.

Many members of the population have difficulty in swallowing solid dosage forms. This is particularly true for the very young, the old and the infirm but can also apply to others particularly if there is not a ready supply of liquid (eg water) to wash down the solid dosage forms.

If the active medicament to be administered has a taste which is perceived by the patient to be unpleasant, then the patient will be less inclined to take the medicament. Several methods of overcoming or masking the taste of unpleasant tasting medicaments have been proposed. Many of these involve coating either the solid dosage form or smaller particles containing the medicament with a material which does not dissolve or disperse in the mouth. Coatings of this type can however slow down the absorption of the active medicament as the coating must be removed before the active medicament can be absorbed either in the stomach or in the gastrointestinal tract.

Another solution to the problem of administering medicines to those who find it difficult to swallow solid dosage forms is to use liquid or gel compositions containing the active medicament. These compositions are not however suitable for everyone. The amount of liquid or gel formulation can vary from dose to dose as the patient or a carer has to dispense an appropriate amount of the composition for example by pouring the composition into a measuring spoon or container. If insufficient care is taken doing this the patient may not be given the intended dose of the active medicament. There is also the possibility that some or all of the intended dose will be spilled before it can be administered, particularly if the patient is reluctant or not in a reasonable physical condition to take the medicine or is uncooperative.

WO 02/03968 is directed to providing a delivery capsule having an enclosing wall comprising a thermoplastic film of foamed (expanded) modified cellulose material. The delivery capsule may contain a unit dose of a throat treatment liquid. The presence of voids in the foamed film results in the film rapidly starting to dissolve in the mouth of a consumer thereby providing a “melt-in-the-mouth” sensation, similar to that of eating rice paper. WO 02/03968 states that this behaviour is to be contrasted to that of non-foamed film of the same thickness which dissolve more slowly in the mouth. Suitably, WO 02/03968 teaches that in order to achieve acceptable dissolution times in the mouth it is necessary to employ a foamed modified cellulose film, as the non-foamed cellulose films are unsuitable for this purpose. It is generally perceived in the art that a foamed film dissolves more rapidly than a corresponding non-foamed film of the same thickness because of the presence of voids in the film.

International patent application PCT/GB02/02637 by The Boots Company PLC is directed to a medicinal composition comprising a core that includes a medicinally effective unit dose of one or more medicaments, wherein the medicaments are enclosed within a film material which comprises at least 40% by weight hydroxypropyl methyl cellulose. Preferably, the core is a “fondant core” comprising a fine crystalline sugar dispersed within a low melting point solid organic carrier.

The present invention seeks to provide a medicinal composition which avoids the problems described above with known solid, semi-solid, liquid and gel dosage forms.

According to a first aspect, the present invention provides a medicinal composition which comprises:
(a) a core in combination with a medicinally effective unit dose of one or more medicaments;
(b) the core being enclosed within a non-expanded film material which comprises a modified cellulose material.

Such a composition may be referred to hereinafter as “the medicinal composition of the present invention”.

Suitably, the core and the encapsulating non-expanded film material typically provides a synergistic effect in that the encapsulating film contains and protects the core and the core supports the film. Unexpectedly, it has been found that a relatively thin non-expanded film material comprising a modified cellulose material may be used to encapsulate the core, which typically dissolves and/or disperses rapidly in the mouth. Suitably, the non-expanded film material is typically less expensive to manufacture than a corresponding expanded film material, as it is not necessary to form voids within the film material, for example by gasifying the film forming mixture. Typically, the non-expanded film material is easier to manipulate and exhibits less faults (e.g. less prone to tear and/or rupture) during processing, compared with an expanded film material. Consequently, the medicinal composition of the present invention is typically cheaper and more easily manufactured than a corresponding composition including an expanded modified cellulose film material. Conveniently, the non-expanded film material may exhibit improved aesthetic and tactile properties compared with an expanded film, which may increase patient compliance for the medicinal composition of the present invention.

Suitably, as the medicament in the medicinal composition of the present invention is enclosed with the film material, a solid, semi-solid, liquid or gel formulation of medicament may be administered to a patient without the need for the patient or a carer to dispense an appropriate amount of the formulation. Conveniently, the medicinal composition of the present invention typically permits administration of an accurate dosage of a medicament to a patient.

Preferably, the medicinal composition of the present invention does not include a core that is a fondant core as disclosed in International patent application PCT/GB02/02637. In other words, the medicinal composition of the present invention does not include a core comprising a fine crystalline sugar dispersed within a low melting point solid organic carrier. For example, a solid organic carrier having a melting point in the range 22 to 60° C., preferably 25 to 40° C., more preferably 32 to 34° C. Examples of suitable low melting point solid organic carriers as disclosed in PCT/GB02/02637 include hydrogenated coconut oil; polyethylene glycols, for example selected from PEG 1000, PEG 2000 and PEG 3000 ranges of polyethylene glycols; povidone and gelucire.

By the term “non-expanded film material”, it is meant the material is essentially free of small openings, pockets or voids within the body of the material. Suitably, the non-expanded film material has a void volume which represents less than or equal to 15%, preferably less than or equal to 10%, more preferably less than or equal to 5%, even more preferably less than or equal to 2%, even more preferably less than or equal to 0.5% by volume of the total volume of the non-expanded film material. Most preferably, the non-expanded film material has essentially no void volume. It will be appreciated by those skilled in the art that it is not necessary to gasify the film forming mixture whilst forming a non-expanded film, unlike methods for forming an expanded film. Suitably, the void volume of the film may be determined by initially measuring the density of the film by flotation weight loss in accordance with ASTM D-782 and then calculating the void volume from the density data.

Preferably, the non-expanded film material includes a first major surface having an essentially smooth and/or non-pitted surface texture extending across a part of or the entire surface. More preferably, the first major surface has an essentially smooth and/or non-pitted surface texture which extends over the entire surface. Even more preferably, both of the first and second major surfaces of the non-expanded film material (i.e. the inner surface of the film communicating with the core of the medicinal composition of the present invention and the outer surface of the film) have an essentially smooth and/or non-pitted surface texture extending across a part of or the entire surface of both the first and second major surfaces. Most preferably both of the major surfaces have an essentially smooth and/or non-pitted surface texture which extends over the entire surfaces of both the first and second major surfaces.

By the term “smooth and/or non-pitted” we mean the surface texture is essentially smooth to human touch. Preferably, by the term “smooth and/or non-pitted” we mean the surface has a roughness average (Ra) of less than or equal to 3 μm, more preferably less than or equal to 1 μm, even more preferably less than or equal to 0.2 μm, even more preferably less than or equal to 0.025 μm, most preferably less than or equal to 0.012 μm as measured in accordance with ASME B46.1-1995.

The film typically has a thickness of less than or equal to 300 μm preferably less than or equal to 200 μm more preferably less than or equal to 150 μm, even more preferably less than or equal to 100 μm, most preferably less than or equal to 80 μm. Preferably, the film has a thickness of greater than or equal to 15 μm, more preferably greater than or equal to 20 μm, even more preferably greater than or equal to 30 μm, most preferably greater than or equal to 40 μm. It is desired to use as thin a film as possible in order to provide relatively short dissolution times of the medicinal composition of the present invention in the mouth. It will be appreciated that the thicker the film, the longer the dissolution time will be. Suitably, by the term “film thickness” it is meant the thickness of the film in the ultimate end product. As described herein, the medicinal compositions of the present invention may be formed by using a thermoforming, vacuum forming and/or heat sealing techniques. Suitably, the thickness of the original film may be reduced by up to approximately 40% in the ultimate end product.

By the term “modified cellulose material”, it is meant a synthetic thermoplastic material that is a modified form of the naturally occurring polymer cellulose. Preferably, the modified cellulose derivative comprises hydroxypropylmethyl cellulose (HPMC), hydroxypropyl cellulose (HPC), methyl cellulose, hydroxyethyl cellulose or a combination of two or more of these modified cellulose derivatives. More preferably the modified cellulose derivative comprises HPMC and/or HPC, particularly HPMC. Preferred HPMCs have 19 to 30% methoxyl substitution and 4 to 12% hydroxypropyl substitution, particularly 28% to 30% methoxyl substitution and 7 to 10% hydroxypropyl substitution. An especially preferred HPMC is available from Dow Chemical Company and sold under the trademark Methocel E50.

Preferably, the non-expanded film material comprises greater than or equal to 40% by wt, more preferably greater than or equal to 50% by wt, even more preferably greater than or equal to 60% by wt, most preferably greater than or equal to 70% by wt, based on the total weight of the film material, of a modified cellulose material as defined herein, in particular HPMC.

Preferably, the non-expanded film material comprises less than or equal to 100% by wt, more preferably less than or equal to 95% by wt, even more preferably less than or equal to 90% by wt, most preferably less than or equal to 85% by wt, based on the total weight of the film material, of a modified cellulose material as defined herein, in particular HPMC.

In a particularly preferred embodiment, the non-expanded film material includes one or more plasticisers. Preferably, the non-expanded film material includes greater than or equal to 5% by wt based on the total weight of the film material, more preferably greater than or equal to 10% by wt, most preferably greater than or equal to 15% by wt of one or more plasticisers. Preferably, the non-expanded film material includes less than or equal to 60% by wt based on the total weight of the film material, more preferably less than or equal to 50% by wt, most preferably less than or equal to 40% by wt of one or more plasticisers. The one or more plasticisers typically provide the film with desired properties, such as improved flexibility. Examples of materials which may be used as plasticisers include polyethylene glycol (PEG), propylene glycols such as monopropylene glycol, glycerol and acetates of glyercol (acetins such as diacetin).

The non-expanded film material may include optional components such as colourants, flavourings, texture modifiers and/or acid materials, such as organic acids. The inclusion of acid materials e.g. citric acid may provide an improved mouth feel for the consumer.

The encapsulating non-expanded film may include an outer coating conveniently used in oral medicaments.

To produce the non-expanded film, the modified cellulose material as defined herein, typically in the form of a powder, is mixed with the plasticiser (if present) and water to produce an aqueous solution. The further components (if present) are then dissolved or dispersed in the solution. Typically, the aqueous solution is stirred e.g. for 6 hours and then left to stand (e.g. 24 to 48 hours) to permit removal of all entrapped air bubbles. A layer of the solution is then cast onto a suitable substrate, e.g. a conveyor belt or glass sheet, and the water removed, e.g. by heating with hot air (e.g. up to 60° C.), to form a dried non-expanded film which is removed from the substrate.

The film is then used to encapsulate a core as described herein. The encapsulation process may use any conventional process, e.g. as disclosed in WO 97/355537, WO 00/27367 or WO 01/03676.

Although the film material is cold water soluble, the resulting capsules are nevertheless found to be sufficiently robust to withstand the production and packaging processes. In addition, they may be held in the hand without the film wall dissolving or rupturing prematurely. However, it will be appreciated that prolonged contact with sweat or other skin secretions may lead to the eventual dissolution of the film wall.

The medicinal compositions in accordance with the present invention are intended to deliver the active medicament(s) carried in the core to the oral cavity or throat of the user. This is particularly useful if the active medicament is intended to treat coughs, sore throats, toothache, or ease respiratory blockages.

In use, the film starts to dissolve almost immediately after introduction into the mouth. The dissolution may be aided by the action of sucking or chewing performed by the user. The film material dissolves completely in the mouth after a short time and typically leaves no unpleasant residues. The dissolution time is dependent upon the film thickness, but is usually less than one minute, typically less than 30 seconds and possibly even quicker e.g. less than 15 seconds, or only a few seconds.

Thus, the medicinal compositions of the present invention are intended to be ruptured in the mouth of the user for release of the core into the mouth. In other words, the compositions of the present invention comprise an edible delivery vehicle.

The medicinal composition of the present invention may be in the form of a capsule having a range of different sizes and shapes depending on the intended use. The capsules may be spherical, ovoid or hemi-spherical in cross-section. Typically, the maximum dimension of the capsule is in the range of 3 mm to 50 mm.

Preferably, the core in the medicinal composition of the present invention is enclosed essentially totally with a non-expanded film material as defined herein.

It will be appreciated that the core and the active medicament represent different entities. Suitably, the core acts as a carrier for the one or more medicaments. The core may comprise a medicinally acceptable carrier or diluent in addition to the active medicament. Preferably, the core has certain physical characteristics which enable it to provide the non-expanded film material with a desired degree of support.

In particular, the core as defined herein may have a physical form of a viscous liquid, a gel, a semi-solid or a solid at room temperature (20° C. to 25° C.) and pressure. Preferably, the core as defined herein is in the form of a viscous liquid or gel, or semi-solid at room temperature and pressure. The active medicament may be dissolved, dispersed or suspended in the core.

Preferably, the core as defined herein has a viscosity of at least 10 Pa·s at a sheer stress of 1 Pa measured at 25° C. Desirably, the core viscosity is at least 50 Pa·s, more preferably at least 100 Pa·s. and most preferably at least 1000 Pa·s when measured at 25° C. at a sheer stress of 1.0 Pa. The viscosity may be measured using an AR 2000 Rheometer with a 20 mm cross-hatched steel plate.

In addition, the core as defined herein preferably exhibits a peak normal force of at least 0.1N, more preferably at least 1N, most preferably at least 5N during a squeeze flow test conducted at 25° C. over 500 seconds at a compression rate of 10 μm/sec using a sample disc 4-8 mm in diameter and up to 500 μm thick. The squeeze flow test measures the biaxial extension (squeeze and subsequent rate of movement) of the sample when compressed. The test may be carried out using an AR 2000 Rheometer fitted with 8 mm steel plates.

The core as defined herein dissolves or disperses rapidly in the mouth of a consumer. This preferably occurs within 10 to 90 seconds after exposure of the core to saliva, preferably 20 to 80 seconds, more preferably 30 to 60 seconds. However, in certain circumstances, e.g. where the medicinal composition of the present invention is intended to treat a sore throat, it may be desirable to have a longer dissolution/dispersion time, e.g. up to 300 seconds, to provide a soothing sensation over a longer period of time.

The core as defined herein has the advantage of being in the physical form of a viscous liquid, a gel, a semi-solid or a solid when encapsulated with the non-expanded film material at 20 to 25° C. (ie room temperature). Suitably this provides the non-expanded film with the desired degree of support and typically results in a robust medicinal product. However, once exposed to saliva, the core dissolves and/or disperses to provide the consumer with a desirable “melt in the mouth” feeling. This assists in soothing; for example sore throats and irritating coughs, without the formulation and production problems associated with providing a free-flowing liquid-containing medicament.

Thus the benefits of a free-flowing liquid-containing medicament may be obtained without having to use, for example, a relatively thick and slow dissolving encapsulating film in order to provide the end product with sufficient robustness and strength for it to be commercially acceptable.

Preferably, the core is selected from an aqueous fondant core, an aqueous gel an emulsion or a thickened oil.

By the term “aqueous fondant core”, it is meant a fine crystalline sugar that is dissolved and/or dispersed within an aqueous carrier, in particular water.

Preferably, the sugar is partially dissolved in the aqueous carrier with the remaining sugar present as a fine crystalline sugar dispersed within the fondant core, especially the aqueous carrier.

The fine crystalline sugar component of the aqueous fondant core preferably has a weight average particle size in the range 1-150 μm, more preferably 10-100 μm, and most preferably 10-25 μm, when measured by electron microscopy. The sugar is preferably selected from sucrose, fructose, glucose, trehalose, a partially or fully invert sugar, and combinations of two or more of these sugars, although any suitable sugar could be used. Sugar derivatives may also be used either in addition to the sugar or as an alternative to it, provided that the sugar or sugar derivative has the required particle size properties and is pharmaceutically acceptable.

Preferably, the aqueous fondant core comprises greater than or equal to 50% by weight, more preferably greater than or equal to 55% by weight, even more preferably greater than or equal to 60% by weight, even more preferably greater than or equal to 65% by weight, most preferably greater than or equal to 70% by weight, based on the total weight of the aqueous fondant core, of one or more sugars as defined above.

Preferably, the aqueous fondant core comprises less than or equal to 95% by weight, more preferably less than or equal to 93% by weight, based on the total weight of the aqueous fondant core, of one or more sugars as defined above.

Preferably, the aqueous fondant core comprises less than or equal to 50% by weight, more preferably less than or equal to 45% by weight, even more preferably less than or equal to 40% by weight, even more preferably less than or equal to 35% by weight, most preferably less than or equal to 30% by weight, based on the total weight of the aqueous fondant core, of the aqueous carrier, especially water.

Preferably, the aqueous fondant core comprises greater than or equal to 5% by weight, more preferably greater than or equal to 7% by weight based on the total weight of the aqueous fondant core, of the aqueous carrier, especially water.

Preferably, the weight to weight ratio of the one or more sugars to the aqueous phase in the aqueous fondant core is greater than or equal to 1:1, more preferably greater than or equal to 1:3, even more preferably greater than or equal to 1:5.

Suitably, when the core comprises an aqueous fondant, the core may be formed in situ by boiling an aqueous sugar solution to form a concentrated mixture, followed by further cooling the concentrated mixture with agitation (ie beating) to produce the aqueous fondant having the fine crystalline sugar dispersed therein. The one or more medicaments may then be dissolved, dispersed or suspended within the core by mixing the medicament(s) with the aqueous fondant core.

Alternatively, the fine crystalline sugar may be mixed directly with the aqueous phase and the resulting solution mixed until the fine crystalline sugar is evenly dispersed therein. The one or more medicaments may be mixed directly with the aqueous phase and crystalline sugar prior to forming the aqueous fondant core. Alternatively, or additionally, the one or more medicaments may be mixed with the aqueous fondant core itself.

Preferably, the aqueous fondant core is in the form of a viscous liquid, for example a syrup, a gel, semi-solid or solid at room temperature and pressure. Suitably, the aqueous fondant core dissolves and/or disperses once exposed to saliva thereby providing the consumer with a desirable “melt in the mouth” feeling. Preferably, the aqueous fondant core has a viscosity and exhibits a peak normal force in the range as defined herein.

Conveniently, the viscosity of the aqueous fondant core may be modified by the inclusion of one or more thickening or viscosity modifying agents. Examples of suitable thickening or viscosity modifying agents for aqueous fondant cores include acacia, alginic acid, carboxymethyl cellulose, cellulose, dextrin, ethyl cellulose, gelatin, glucose, guar gum, hydroxyethyl cellulose, hydroxypropylmethyl cellulose, liquid glucose, magnesium aluminium silicate, maltodextrin, methyl cellulose, polyethylene oxide, polymethacrylates, penidone, sodium alginate, starch, vegetable oil, zein, gum tragacanth, gum acacia, xanthan gum, egg albumin, pectin, carrageenan or gellan gum. The amount of thickening or viscosity modifying agents may be in the range of 0.5 to 30% w/w, preferably 0.5 to 20% w/w more preferably 0.5 to 10% w/w, most preferably 0.5 to 5% w/w based on the total weight of the core.

A highly preferred aqueous fondant core comprises, preferably consists essentially of, 50 to 95% by weight of one or more sugars and 5 to 50% by weight water.

By the term “aqueous gel”, it is meant an aqueous phase, in combination with a thickening or viscosity modifying agent such that the mixture has the consistency of a viscous liquid, syrup or gel. Preferably the aqueous phase for the aqueous gel is selected from water or an aqueous sugar mixture.

Examples of suitable thickening or viscosity modifying agents for the aqueous gel core include those listed above in relation to the aqueous fondant core.

Preferably, the aqueous gel core comprises greater than or equal to 70% by weight, more preferably greater than or equal to 75% by weight, even more preferably greater than or equal to 80% by weight, more preferably greater than or equal to 85% by weight, based on the total weight of the aqueous gel core, of the aqueous phase as defined herein.

Preferably the aqueous gel core comprises less than or equal to 30% by weight, more preferably less than or equal to 25% by weight, even more preferably less than or equal to 20% by weight, more preferably less than or equal to 15% by weight, based on the total weight of the aqueous gel core, one or more thickening or viscosity modifying agents as defined herein.

Preferably, the aqueous gel core comprises less than or equal to 99% by weight, more preferably less than or equal to 98% by weight, even more preferably less than or equal to 97% by weight, based on the total weight of the aqueous gel core, of the aqueous phase as defined herein.

Preferably, the aqueous gel core comprises greater than or equal to 1% by weight, more preferably greater than or equal to 2% by weight, even more preferably greater than or equal to 3% by weight, based on the total weight of the aqueous gel core, of one or more thickening or viscosity modifying agents as defined herein.

Preferably, the weight to weight ratio of the aqueous phase to the one or more thickening or viscosity modifying agents is greater than or equal to 3:1, more preferably greater than or equal to 4:1, even more preferably greater than or equal to 5:1. A highly preferred range of weight to weight ratios of the aqueous phase to the one or more thickening or viscosity modifying agents is 4:1 to 25:1.

As mentioned herein, the aqueous phase of the aqueous gel core may consist essentially of water or it may comprise an aqueous sugar mixture. Suitably, the aqueous sugar mixture comprises one or more sugars dissolved in an aqueous carrier, in particular water. Suitable sugars are those sugars as defined herein in respect of the “aqueous fondant core” and preferably such sugars have the same physical properties i.e. weight average particle size distribution.

Preferably, the aqueous sugar mixture comprises greater than or equal to 50% by weight, more preferably greater than or equal to 55% by weight, most preferably greater than or equal to 60% by weight, based on the total weight of the aqueous sugar mixture, of one or more sugars as defined herein.

Preferably, the aqueous sugar mixture comprises less than or equal to 80% by weight, more preferably less than or equal to 75% by weight, most preferably less than or equal 70% by weight, based on the total weight of the aqueous sugar mixture, of one or more sugars as defined herein.

Preferably the aqueous sugar mixture comprises greater than or equal to 20% by weight, more preferably greater than or equal to 25% by weight, more preferably greater than or equal to 30% by weight, based on the total weight of the aqueous sugar mixture, of the aqueous carrier, especially water.

Preferably, the aqueous sugar mixture comprises less than or equal to 50% by weight, more preferably less than or equal to 45% by weight, most preferably less than or equal to 40% by weight, based on the total weight of the aqueous sugar mixture, of the aqueous carrier, especially water.

Suitably, the aqueous gel core may be produced by mixing, preferably at room temperature, the aqueous phase and the one or more thickening or viscosity modifying agents. Suitably, the one or more medicaments may be added prior to formation of the aqueous gel core and/or to the aqueous gel core itself.

Preferably, the aqueous gel core has a viscosity and exhibits a peak normal force in the range as defined herein. As mentioned, the aqueous gel may have the consistency of a viscous liquid, syrup or gel at room temperature and pressure. Suitably, the aqueous gel core has a desirable mouth feel and it disperses on contact with saliva to provide the consumer with a desirable free-flowing liquid-containing medicament feeling.

A highly preferred aqueous gel core comprises, preferably consists essentially of, 70 to 99% by weight of the aqueous phase as defined herein and 1 to 30% by weight of one or more thickening or viscosity modifying agents as defined herein.

By the term “thickened oil”, it is meant an oil in combination with a thickening or viscosity modifying agent. Suitably, the oil is a liquid at room temperature, it has a melting point of less than 20° C. and it is medicinally acceptable for ingestion by a human or animal.

Suitable oils include mono-, di- and tri-glyceride derivatives of one or more fatty acids. Suitably, one or more fatty acids include short chain (C₆ to C₁₀), medium chain (C₁₂ to C₁₄) and long chain (C₁₆ or more) fatty acids. Preferably, the one or more fatty acids include C₈ to C₂₀ fatty acids, in particular C₈ to C₁₈ fatty acids. Suitably, the one or more fatty acids may be straight chain or branched.

Preferably, the one or more fatty acids are straight chain. Suitably, the one or more fatty acids may be saturated or unsaturated. Highly preferred fatty acids include caprylic acid, decanoic acid, myristic acid, palmitic acid, oleic acid, stearic acid and combinations of two or more of these acids. Highly preferred oils include mono-, di- and tri-glyceride derivatives which contain greater than or equal to 70%, more preferably greater than or equal to 80%, most preferably greater than or equal to 90% of one or more saturated fatty acids as defined herein.

Suitably, the oil may be derived from an animal or vegetable source and may include natural, refined and/or synthetically modified oils derived from such sources. Suitable oils derived from such sources include olive, lucca, rapeseed, sunflower, soyabean, arachis, peanut, ground nut, coconut, maize, corn flower and fish oils.

Suitable oils include sunflower oils and rapeseed oils having a high oleic acid content which are available from Karlshamns. An especially preferred oil is Miglyol available from Hüls which comprises a mixture of triglycerides where greater than 95% of the fatty acids are the saturated fatty acids caprylic acid and decanoic acid.

Preferably, the thickened oil core comprises greater than or equal to 50% by weight, more preferably greater than or equal to 55% by weight, even more preferably greater than or equal to 60% by weight, most preferably greater than or equal to 65% by weight, based on the total weight of the thickened oil core, of one or more oils as defined above.

Preferably, the thickened oil core comprises less than or equal to 99% by weight, more preferably less than or equal to 98% by weight, even more preferably less than or equal to 95% by weight, most preferably less than or equal to 90% by weight, based on the total weight of the thickened oil core, of one or more oils as defined above.

The thickened oil core includes one or more thickening or viscosity modifying agents. Suitable viscosity modifying or thickening agents for the thickened oil core include colloidal silicon dioxide, glycerol monostearate or a mixture of these thickening agents.

Preferably, the thickened oil core comprises less than or equal to 50% by weight, more preferably less than or equal to 45% by weight, even more preferably less than or equal to 40% by weight, most preferably less than or equal to 35% by weight, based on the total weight of the thickened oil core, of one or more thickening or viscosity modifying agents.

Preferably the thickened oil core comprises greater than or equal to 1% by weight, more preferably greater than or equal to 2% by weight, even more preferably greater than or equal to 5% by weight, most preferably greater than or equal to 10% by weight, based on the total weight of the thickened oil core, of one or more thickening or viscosity modifying agents.

Preferably, the weight to weight ratio of the one or more oils to the one or more thickening or viscosity modifying agents in the thickened oil core is greater than or equal to 2:1, more preferably greater than or equal to 3:1, most preferably greater than or equal to 4:1.

Optionally, the thickened oil core may include one or more fine crystalline sugars dispersed or suspended in the oil phase of the core. Suitable sugars are those sugars as defined herein in respect of the “aqueous fondant core” and preferably such sugars have the same physical properties i.e. weight average particle size distribution.

When a sugar is present in the thickened oil, preferably the sugar is present in the range of 5 to 70% by weight, more preferably 10 to 60% by weight, most preferably 20 to 50% by weight based on the total weight of the oil in the thickened oil core.

Preferably, the thickened oil core has a viscosity and/or exhibits a peak normal force in the range as defined herein. It will be appreciated that the viscosity of the thickened oil core may be modified by varying the amount of thickening or viscosity modifying agent in the core. Typically, the thickened oil is in the form of a viscous liquid, a semi-solid or gel at room temperature and pressure. Suitably, the active medicament is dissolved, dispersed or suspended in the core. Suitably, the thickened oil core has a desirable mouth feel and it disperses on contact with saliva to provide the consumer with a desirable free-flowing liquid-containing medicament feeling.

Suitably, when the core comprises a thickened oil, the core may be formed by combining the oil and the thickening or viscosity modifying agent and sugar (if present) with stirring, optionally with heating. The one or more medicaments may be added prior to formation the core and/or to the core itself.

A highly preferred thickened oil core comprises, preferably consists essentially of, 50 to 99% by weight of one or more oils as defined herein and 1 to 50% by weight of one or more thickening or viscosity modifying agents as defined herein.

Suitably, the emulsion core may be an oil-in-water type emulsion, a water-in-oil type emulsion or a non-aqueous type emulsion.

Suitably, the oil-in-water type emulsion comprises an external aqueous phase and an internal oil phase. Preferably the aqueous phase consists essentially of an aqueous sugar mixture as defined herein in respect of the “aqueous gel core”, alternatively the aqueous phase consists essentially of the “aqueous gel core” as defined herein. Preferably, the aqueous phase consists of an aqueous sugar mixture. Preferably, the aqueous phase contains substantially no free or unbound water as defined herein.

Preferably, the aqueous phase in the oil-in-water type emulsion is present in an amount of greater than 50% by weight, more preferably greater than or equal to 55% by weight, most preferably greater than or equal to 60% by weight of the oil-in-water type emulsion.

Preferably, the aqueous phase of the oil-in-water type emulsion is present in an amount of less than or equal to 90% by weight, more preferably less than or equal to 85% by weight, most preferably less than or equal to 80% by weight of the oil-in-water type emulsion.

Preferably, the oil phase in the oil-in-water type emulsion is present in an amount of greater than or equal to 10% by weight, more preferably greater than or equal to 15% by weight, most preferably greater than or equal to 20% by weight of the oil-in-water emulsion.

Preferably, the oil phase in the oil-in-water type emulsion is present in an amount of less than 50% by weight, more preferably less than or equal to 45% by weight, most preferably less than or equal to 40% by weight of the oil-in-water emulsion.

Preferably, the weight to weight ratio of the aqueous phase to the oil phase in the oil-in-water type emulsion is greater than 1:1, more preferably greater than or equal to 2:1.

Suitably, the water-in-oil type emulsion comprises an external oil phase and an internal aqueous phase. Suitably the internal aqueous phase consists essentially of water, an aqueous sugar mixture as defined herein in respect of the “aqueous gel core”, or the “aqueous gel core” itself as defined herein.

Preferably, the oil phase in the water-in-oil type emulsion is present in an amount of greater than 50% by weight, more preferably greater than or equal to 55% by weight, most preferably greater than or equal to 60% by weight of the water-in-oil emulsion.

Preferably, the oil phase in the water-in-oil type emulsion is present in an amount of less than or equal to 90% by weight, more preferably less than or equal to 85% by weight, most preferably less than or equal to 80% by weight of the water-in-oil emulsion.

Preferably, the aqueous phase in the water-in-oil type emulsion is present in an amount of greater than or equal to 10% by weight, more preferably greater 15% by weight, most preferably greater than or equal to 20% by weight of the water-in-oil emulsion.

Preferably, the aqueous phase in the water-in-oil type emulsion is present in an amount of less than 50% by weight, more preferably less than or equal to 45% by weight, most preferably less than or equal to 40% by weight of the oil-in-water emulsion.

Preferably, the weight to weight ratio of the oil phase to the aqueous phase in the water-in-oil type emulsion is greater than 1:1, more preferably greater than or equal to 2:1.

Suitable oils which may be employed as the oil phase in the oil-in-water and water-in-oil type emulsions include one or more oils derived from an animal or vegetable source and may include natural, refined and/or synthetically modified oils derived from such sources. Preferably, the one or more oils include those oils as defined in respect of the “thickened oil core”.

Alternatively, or additionally, the one or more oils may include a low melting point fat. Preferably, the low melting point fat has a melting point in the range 22 to 60° C., preferably 25 to 40° C., more preferably 32 to 34° C. Examples of such fats include hydrogenated coconut oil, cocoa butter, and butter fat. It will be appreciated that when one or more low melting point fats are present, the sugar is dissolved in the aqueous phase. Hence, the resulting emulsion does not include one or more fine crystalline sugars dispersed within the low melting point fat.

Preferably, the one or more oils consists essentially of those oils as defined in respect of the “thickened oil core”.

Suitably, the oil-in-water type emulsion and water-in-oil type emulsion may be formed by preparing the oil phase and aqueous phase separately. Mixing the oil phase and the aqueous phase together, typically with high shear mixing. The emulsifier (if present) is added to whichever phase it is miscible/soluble with. The one or more medicaments may be added to the mixture prior to or during formation of the emulsion or to the emulsion per se.

Suitably, the non-aqueous type emulsion consists essentially of the oil-in-water type emulsion or the water-in-oil type emulsion as defined herein except the aqueous phase of such emulsion(s) has been replaced with a polar organic phase. Preferably, the polar organic phase is present in the non-aqueous emulsion in an amount identical to that of the aqueous phase in the oil-in-water type emulsion or water-in-oil type emulsion, respectively.

Preferably, the polar organic phase in the non-aqueous emulsion is selected from glycerin, polyethylene glycols, propylene glycols and mixtures thereof.

The non-aqueous emulsion core may be formed in an analogous manner to the water-in-oil and oil-in-water type emulsions.

Optionally, the emulsions as defined herein may include an emulsifier, such as lecithin, esters of sorbitan, and glyceryl monostearate. Suitably, the emulsifier may be present in an amount of 0.1 to 25% by weight, preferably 0.25 to 15% by weight based on the total weight of the major phase of the emulsion.

Preferably, the emulsions as defined herein have a viscosity and/or exhibit a peak normal force in the range as defined herein. Suitably, the emulsion is in the form of a viscous liquid, gel or semi-solid. Suitably, the emulsions have a desirable mouth feel and disperse on contact with saliva to provide the consumer with a desirable free-flowing liquid-containing medicament feeling.

Suitably, the core as defined herein may include other excipients. The other excipients may include taste masking agents, artificial sweeteners, flavours, inert diluents, and lubricants.

Suitable taste masking agents are described hereinafter.

Suitable artificial sweeteners include acesulfame K, sodium saccharin, aspartame. The amount of sweetener may be in the range 0.001% to 2%.

Suitable flavours are commercially available and may be enhanced by the addition of an acid, for example citric acid, ascorbic acid, tartaric acid.

Suitable inert diluents include calcium phosphate (anhydrous and dihydrate), calcium sulphate, carboxymethylcellulose calcium, cellulose acetate, dexrates, dextrin, dextrose, fructose, glyceryl palmitostearate, hydrogenated vegetable oil, kaolin, lactitol, lactose, magnesium carbonate, magnesium oxide, maltitol, maltodextrin, maltose, microcrystalline cellulose, polymethacrylates, powdered cellulose, pregelatinised starch, silicified microcrystalline cellulose, sodium chloride, starch, sucrose, sugar, talc, xylitol. One or more diluents may be used. The amount of diluent may be in the range 10-98% w/w.

Suitable lubricants include calcium stearate, canola oil, glyceryl palmitostearate, hydrogenated vegetable oil, magnesium stearate, mineral oil, poloxamer, polyethylene glycol, polyvinyl alcohol, sodium benzoate, sodium lauryl sulphate, sodium stearyl fumarate, stearic acid, talc and zinc stearate. One or more lubricants may be used. The lubricant may be in the range 0.01-10% w/w.

The core preferably contains substantially no free or unbound water. This is because the non-expanded film material of the capsule shell is cold water soluble. However, bound water, e.g. present as part of a carbohydrate solution such as a syrup, is acceptable, up to levels of about 40% by weight of the core. By “substantially no free or unbound water”, it is meant that the core preferably contains less than 1% by weight free or unbound water, more preferably less than 0.1% by weight, even more preferably less than 0.05% by weight and most preferably 0% by weight free or unbound water.

The active medicament may be an analgesic or anti-inflammatory, decongestant, cough suppressant, expectorant, mucolytic, antihistamine, antiallergy agent, agent for treating the gastrointestinal tract (for example antacid, antireflux agent, antiulcer agent, antidiarrhoeal agent, laxative or antiemetic), agent to counter motion sickness, antiviral agents, antifungal agents, antibacterial agents, diuretic agents, antiasthmatic agents, antimigraine agents, antianxiety agents, tranquilising agents, sleep promoting agents, vitamins and/or minerals, natural products and extracts thereof (for example herbs or naturally occurring oils)

Suitable analgesics include aspirin, paracetamol (acetaminophen) and non-steroidal anti-inflammatory/analgesics such as diclofenac, indomethacin, mefanamic acid, nabumetone, tolmetin, piroxicam, felbinac, diflunisal, ibuprofen, flurbiprofen, naproxen and ketoprofen, active isomers thereof or medicinally acceptable salts thereof (for example the sodium or lysine salts) or narcotic analgesics such as codeine and medicinally acceptable salts thereof (for example codeine phosphate or sulphate). Caffeine may be present in analgesic products to enhance the analgesic effect

The amount of aspirin in a unit dose may be in the range 75 to 800 mg, preferably 200-600 mg, most preferably 75, 150, 300, 400 or 600 mg. The amount of paracetamol in a unit dose may be 50 to 2000 mg, preferably 120 to 1000 mg, most preferably 120, 250, 500 or 1000 mg. The amount of diclofenac in a unit dose may be 10 to 100 mg, preferably 20 to 80 mg, most preferably 25 or 50 mg. The amount of indomethacin in a unit dose may be in the range 25-75 mg, preferably 25 mg, 50 mg or 75 mg. The amount of mefanamic acid in a unit dose may be in the range 250-500 mg, preferably 250 mg or 500 mg. The amount of nabutmetone in a unit dose may be in the range 500-1000 mg. The amount of piroxicam in a unit dose may be in the range 10-40 mg, preferably 10, 20 or 40 mg. The amount of diflunisal in a unit dose may be in the range 250-500 mg, preferably 250 mg or 500 mg. The amount of ibuprofen in a unit dose may be in the range 50 to 800 mg, preferably 100 to 400 mg, most preferably 100, 200 or 400 mg. The amount of flurbiprofen in a unit dose may be 5 to 200 mg, preferably 5 to 150 mg, most preferably 50 or 100 mg. The amount of naproxen in a unit dose may be 100 to 800 mg, preferably 200 to 600 mg, most preferably 250, 373 or 500 mg. The amount of ketoprofen in a unit dose may be 25 to 250 mg, preferably 50 to 150 mg, most preferably 50 or 100 mg. The amount of codeine in a unit dose may be 20 to 50 mg, preferably 5 to 30 mg, most preferably 8, 12.5, 16 or 25 mg. If medicinally effective salts of the above compounds are used then the amount of salt should be increased to give a dose of the free medicament corresponding to the figures given above. The amount of caffeine in a unit dose may be 5 to 200 mg, preferably 10 to 100 mg, most preferably 30, 45, 60 or 100 mg.

Suitable decongestants include ephedrine, levomethol, pseudoephedrine preferably as its hydrochloride, phenylpropanolamine preferably as its hydrochloride and phenylephrine.

The amount of ephedrine in a unit dose may be in the range 15-60 mg. The amount of levomethol in a unit dose may be in the range 0.5-100 mg, preferably 0.5-25 mg, most preferably 1, 2, 5, 10 or 25 mg. The amount of pseudoephedrine preferably as its hydrochloride in a unit dose may be in the range 60-120 mg, preferably 30, 60 or 120 mg. The amount of phenylpropanolamine preferably as its hydrochloride in a unit dose may be in the range 5-50 mg, preferably 5-20 mg. The amount of phenylephrine in a unit dose may be in the range 5-25 mg, preferably 5, 10 or 25 mg.

Suitable cough suppressants include bibenzonium preferably as its bromide, caramiphen, carbetapentane preferably as its citrate, codeine, dextromethorphan preferably as its hydrobromide or an absorbate thereof, noscapine and pholcodine.

The amount of bibenzonium bromide in a unit dose may be in the range 20-30 mg. The amount of caramiphen in a unit dose may be in the range 5-20 mg, preferably 5 or 20 mg. The amount of carbetapentane citrate in a unit dose may be in the range 15-30 mg. The amount of codeine in a unit dose maybe in the range 2-50 mg, preferably 5-30 mg, most preferably 10 mg. In the present invention medicinally acceptable salts of codeine may also be used (for example codeine phosphate or sulphate). The amount of dextromethorphan hydrobromide in a unit dose may be in the range 5-60 mg, preferably 15 or 30 mg. The amount of noscapine in a unit dose may be in the range 15-30 mg. The amount of pholcodeine in a unit dose may be in the range 2-25 mg, preferably 5 to 20 mg, more preferably 10 to 15 mg.

Suitable expectorants include ammonium bicarbonate, ammonium chloride, bromhexine hydrochloride, cocillana creosote, gualfenesin, ipecacuanha, potassium and medicinally acceptable salts thereof (for example potassium citrate or iodide), potassium guaicolsulfonate, squill and terpin hydrate.

The amount of ammonium bicarbonate in a unit dose may be in the range 300-600 mg. The amount of ammonium chloridein in a unit dose may be in the range 0.3-2 g (300-2000 mg). The amount of bromhexine hydrochloride in a unit dose may be in the range 24-64 mg. The amount of cocillana creosote in a unit dose may be in the range 0.12-0.6 ml. The amount of gualfenesin in a unit dose may be in the range 100-200 mg, preferably 100 mg. The amount of ipecacuanha in a unit dose may be in the range 25-100 mg. The amount of potassium iodide in a unit dose may be in the range 150-300 mg, preferably 100 mg. The amount of potassium citrate in a unit dose may be in the range 150-300 mg, preferably 100 mg. The amount of potassium guaicolsulfonate in a unit dose may be 80 mg. The amount of squill in a unit dose may be in the range 60-200 mg. The amount of terpin hydrate in a unit dose may be in the range 125-600 mg, preferably 300 mg.

Suitable mucolytic agents include ambroxyl, acetylcystine and carbocisteine

The amount of carbocisteine in a unit dose may be in the range 100 mg to 1000 mg, preferably 200 to 500 mg

Suitable antihistamines include azatadine or a salt thereof such as the maleate, bromodiphenhydramine or a salt thereof such as the hydrochloride, brompheniramine or a salt thereof such as the maleate, carbinoxamine or a salt thereof such as the maleate, chlorpheniramine or a salt thereof such as the maleate, cyproheptadine or a salt thereof such as the hydrochloride, dexbrompheniramine or a salt thereof such as the maleate, dexchlorpheniramine or a salt thereof such as the maleate, diphenhydramine or a salt thereof such as the hydrochloride, doxylamine or a salt thereof such as the succinate, phenidamine or a salt thereof such as the tartrate, promethazine or a salt thereof such as the hydrochloride, pyrilamine or a salt thereof such as the maleate, pyrilamine or a salt thereof such as the tannate, tripelennamine or a salt thereof such as the hydrochloride, tripolidine or a salt thereof such as the hydrochloride, cetirizine or a salt thereof such as the hydrochloride, cinnarizine, mequitazine, dcivastine.

The amount of azatadine in the form of maleate in a unit dose may be in the range 1-2 mg, preferably 1 mg. The amount of bromodiphenhydramine in the form of hydrochloride in a unit dose may be 3.75 mg. The amount of brompheniramine in the form of maleate in a unit dose may be in the range 4-12 mg, preferably 4, 8 or 12 mg. The amount of carbinoxamine in the form of maleate in a unit dose may be 4 mg. The amount of chlorpheniramine in the form of maleate in a unit dose may be in the range 2-12 mg, preferably 4, 8 or 12 mg. The amount of dexbrompheniramine in the form of maleate in a unit dose may be 6 mg. The amount of dexchlorpheniramine in the form of maleate in a unit dose may be in the range of 2-6 mg, preferably 2, 4 or 6 mg. The amount of diphenhydramine in the form of hydrochloride in a unit dose may be in the range of 12.5 to 200 mg, preferably 12.5-50 mg, more preferably 12.5, 25 or 50 mg. The amount of doxylamine in the form of succinate in a unit dose may be in the range 7.5-10 mg, preferably 7.5 or 10 mg. The amount of phenidamine in the form of tartrate in a unit dose may be in the range 5-10 mg, preferably 5 or 10 mg. The amount of promethazine in the form of hydrochloride in a unit dose may be in the range 1.5-6 mg. The amount of pyrilamine in the form of maleate in a unit dose may be 12.5 mg. The amount of pyrilamine in the form of tannate in a unit dose may be 12.5 mg. The amount of tripelennamine in the form of hydrochloride in a unit dose may be in the range 25-50 mg, preferably 25, 37.5 or 50 mg. The amount of triprolidine in the form of hydrochloride in a unit dose may be in the range 1-2.5 mg, preferably 1.25-2.5 mg, most preferably 1.25 mg. The amount of cetrizine in a unit dose may be in the range 5-10 mg, preferably 5 mg or 10 mg. The amount of cinnarizine in a unit dose may be in the range of 15-75 mg, preferably 15 mg or 75 mg. The amount of mequitazine in a unit dose may be in the range 5-10 mg, preferably 5 mg or 10 mg. The amount of acrivastine in a unit dose may be 3-20 mg, preferably 5-10 mg, most preferably around 8 mg.

Suitable antiallergy agents include astemizole, clemastine or a salt thereof such as the hydrogen fumerate, loratadine, terfenadine.

The amount of astemizole in a unit dose may be in the range 0.5-200 mg, preferably 1-100 mg, most preferably 2, 5, 10, 20 or 40 mg. The amount of clemastine in the form of its hydrogen fumerate in a unit dose may be in the range 0.01-200 mg, preferably 0.1-10 mg, most preferably 0.2, 0.4, 0.6, 1.2 or 2.4 mg. The amount of loratadine in a unit dose may be in the range 0.5-200 mg, preferably 1-100 mg, most preferably 2, 5, 10, 20 or 40 mg. The amount of terfenadine in a unit dose may be in the range 5-1000 mg, preferably 10-600 mg, most preferably 20, 40, 60, 100 or 200 mg.

Suitable antacids include aluminium glycinate, aluminium hydroxide gel, aluminium phosphate gel, dried aluminium phosphate gel, calcium carbonate, charcoal, hydrotalcite, light kaolin, magnesium carbonate, magnesium hydroxide, magnesium oxide, magnesium trisilicate, sodium bicarbonate.

The amount of aluminium glycinate in a unit dose may be in the range 0.1-10 g, preferably 0.1-5 g, most preferably 0.2, 0.5, 1 or 2 g. The amount of aluminium hydroxide gel in a unit dose may be in the range 1-50 ml, preferably 2-30 ml, most preferably 5, 7.5, 10, 15 or 30 ml. The amount of aluminium phosphate gel in a unit dose may be in the range 0.5-100 ml, preferably 1-50 ml, most preferably 2, 5, 10, 15 or 30 ml. The amount of dried aluminium phosphate gel in a unit dose may be in the range 50-5000 mg, preferably 100-2000 mg, most preferably 200, 400, 800 or 1600 mg. The amount of calcium carbonate in a unit dose may be in the range 0.1-30 g, preferably 0.5-10 g, most preferably 0.5, 1, 2 or 5 g. The amount of charcoal in a unit dose may be in the range 1-200 g, preferably 1-100 g, most preferably 2, 4, 8, 16 or 50 g. The amount of hydrotalcite in a unit dose may be in the range 0.1-10 g, preferably 0.2-5 g, most preferably 0.5, 1 or 2 g. The amount of light kaolin in a unit dose may be in the range 10 mg-100 g, preferably 100 mg-75 g, most preferably 1, 10, 15, 20, 50 or 75 g. The amount of magnesium carbonate in a unit dose may be in the range 50 mg-10 g, preferably 50 mg-5 g, most preferably 100, 200 or 500 mg. The amount of magnesium hydroxide in a unit dose may be in the range 100 mg-10 g, preferably 100 mg-5 g, most preferably 100, 250, 500 or 750 mg. The amount of magnesium oxide in a unit dose may be in the range 100 mg-10 g, preferably 100 mg-5 g, most preferably 100, 250, 500 or 750 mg. The amount of sodium bicarbonate in a unit dose may be in the range 0.1-50 g, preferably 0.5-25 g, most preferably 0.5, 1, 2, 5 or 10 g.

Suitable antireflux agents include simethicone and sodium alginate.

The amount of simethicone in a unit dose may be in the range 5-1000 mg, preferably 10-500 mg, most preferably 25, 40, 50, 60, 100 or 200 mg. The amount of sodium alginate in a unit dose may be in the range 50 mg-10 g, preferably 75 mg-5 g, most preferably 100, 250, 500 or 1 g.

Suitable antiulcer agents include bismuth subsalicylate, H₂ receptor antagonists such as cimetidine, famotidine, ranitidine and nizatidine and proton pump inhibitors such as omeprazole, pantoprazole and lansoprazole.

The amount of bismuth subsalicylate in a unit dose may be in the range 250-2000 mg, preferably 50-1500 mg, most preferably 75, 150, 300, 600 or 1000 mg. The amount of cimetidine in a unit dose may be in the range 10 mg-5 g, preferably 50 mg-2 g, most preferably 100, 200 or 400 mg. The amount of famotidine in a unit dose may be in the range 10-80 mg, preferably 20 or 40 mg. The amount of ranitidine in a unit dose may be in the range 100-600 mg, preferably 300-600 mg, most preferably 300 or 600 mg. The amount of nizatidine in a unit dose may be 50 to 500 mg, preferably 100 to 400 mg, more preferably 150 to 300 mg. The amount of omeprazole in a unit dose may be 5 to 50 mg, preferably 10 to 40 mg, more preferably 10, 20 or 40 mg. The amount of pantoprazole in a unit dose may be 10 to 50 mg, preferably 15 to 45 mg, more preferably 20 to 40 mg. The amount of lansoprazole in a unit dose may be 5 to 50 mg, preferably 10 to 40 mg, more preferably 15 or 30 mg.

Suitable antidiarrhoeal agents include loperamide or a salt thereof, such as the hydrochloride, methylcellulose, diphenoxylate and morphine or a salt thereof, such as the hydrochloride.

The amount of loperamide in the form of its hydrochloride in a unit dose may be in the range 0.1-50 mg, preferably 0.5-20 mg, most preferably 1, 2, 4 or 8 mg. The amount of methylcellulose in a unit dose may be in the range 20 mg-5 g, preferably 50 mg-4 g, most preferably 100, 200, 500 mg, 1 or 2 g. The amount of diphenoxylate in the form of its hydrochloride in a unit dose may be 1-10 mg, preferably 2-5 mg, more preferably 2.5 mg. The amount of morphine in the form of its hydrochloride in a unit dose may be in the range 20-4000 μg, preferably 50-2000 μg, most preferably 100, 200, 400, 800 or 1600 μg.

Suitable laxatives include agar, aloin, bisacodyl, ispaghula husk, lactulose, phenolphthalein and senna extract (including sennosides A+B).

The amount of agar in a unit dose may be in the range 1-200 mg, preferably 2-100 mg, most preferably 2.5, 5, 10, 20 or 5.0 mg. The amount of aloin in a unit dose may be in the range 1-200 mg, preferably 2-100 mg, most preferably 5, 10, 15 or 30 mg. The amount of bisacodyl in a unit dose may be in the range 0.1-100 mg, preferably 0.5-50 mg, most preferably 1, 2, 5, 10 or 20 mg. The amount of ispaghula husk in a unit dose may be in the range 100 mg-50 g, preferably 500 mg-25 g, most preferably 1, 2, 3, 5 or 10 g. The amount of lactulose in a unit dose may be in the range 100 mg-50 g, preferably 500 mg-30 g, most preferably 1, 2, 5, 10 or 15 g. The amount of phenolphthalein in a unit dose may be in the range 1-5000 mg, preferably 5-4000 mg, most preferably 7.5, 15, 30, 60, 100, 200 or 300 mg. The amount of senna extract (including sennosides A+B) in a unit dose may be in the range 0.5-100 mg, preferably 1-50 mg, most preferably 2.5, 5, 7.5, 10, 15 or 30 mg.

Suitable antiemetics include dimenhydrinate, metoclopromide or a salt thereof such as the hydrochloride, domperidone or a salt thereof such as the maleate, buclizine, cyclizine, prochlorperazine or a salt thereof such as the maleate, ipecacuanha, squill.

The amount of ipecacuanha in a unit dose may be in the range 25-100 mg. The amount of squill in a unit dose may be in the range 60-200 mg. The amount of domperidone may be in the range 5-50 mg, preferably 5, 10, 15, 20, 25, 30, 40 or 50 mg. The amount of buclizine in a unit dose may be in the range 2-100 mg, preferably 5-50 mg, more preferably 6.25, 13.5, 25. The amount of cyclizine in a unit dose may be in the range 1-50 mg, preferably 2-30 mg, more preferably 5, 7.5, 10, 15, 20 or 25 mg. The amount of metoclopromide in a unit dose may be in the range 2-30 mg, preferably 5, 10, 15 or 30 mg. The amount of dimenhydrinate in a unit dose may be in the range 5-50 mg, preferably 25 mg. The amount of prochlorperazine in a unit dose may be in the range 3-25 mg, preferably 3 mg or 5 mg. If medicinally effective salts of the above compounds are used then the amount of salt should be increased to give a dose of the free medicament corresponding to the figures given above.

Suitable agents to counter motion sickness include cinnarizine, dimenhydrinate, hyoscine or a salt thereof such as the hydrobromide and meclozine or a salt thereof such as the hydrochloride.

The amount of cinnarizine in a unit dose may be in the range 0.5-200 mg, preferably 1-100 mg, most preferably 5, 10, 20, 40 or 60 mg. The amount of dimenhydrinate in a unit dose may be in the range 1-500 mg, preferably 5-300 mg, most preferably 10, 20, 50, 100 or 250 mg. The amount of hyoscine hydrobromide in a unit dose may be in the range 0.01-1 mg, preferably 0.05-0.5 mg, most preferably 0.05, 0.1, 0.2, 0.3 or 0.5 mg. The amount of meclozine hydrochloride in a unit dose may be in the range 0.5-200 mg, preferably 1-100 mg, more preferably 2, 5, 10, 20 or 40 mg.

Suitable antiviral agents include aciclovir. The amount of aciclovir in a unit dose may be in the range 100 to 1000 mg, preferably 200 to 800 mg.

Suitable antifungal agents include fluconazole and terbinafine. The amount of fluconazole in a unit dose may be in the range 50-200 mg, preferably 50 mg or 200 mg. The amount of terbinafine may be in the range 250-500 mg, preferably 250 mg.

Suitable antibacterial agents include erythromycin and fusidic acid and salts thereof such as the sodium salt. The amount of erythromycin in a unit dose may be in the range 125-500 mg, preferably 125 mg, 250 mg or 500 mg. The amount of fusidic acid in a unit dose may be in the range 250-500 mg, preferably 250 mg.

Suitable diuretics include frusemide. The amount of frusemide in a unit dose may be in the range 20-80 mg, preferably 20, 40 or 80 mg.

Suitable anti-asthmatic agents include ketotifen. The amount of ketotifen in a unit dose may be in the range 1-4 mg, preferably 1 mg or 2 mg.

Suitable anti-migraine agents include the triptans such as sumatriptan. The amount of sumatriptan in a unit dose may be in the range 20-100 mg, preferably 20, 50 or 100 mg.

Suitable vitamins include A, B1, B2, B3, B5, B6, B12, C, D, E, folic acid, biotin, and K. Suitable minerals include calcium, phosphorus, iron, magnesium, zinc, iodine, copper, chloride, chromium, manganese, molybdenum, nickel, potassium, selenium, boron, tin and vanadium.

The term active medicament as used herein also embraces materials which are known and used to give relief or comfort to a patient even if they have not been shown to have any pharmacological effect. These are referred to hereinafter as “relief agents”. Examples of such materials include anise oil, treacle, honey, liquorice and menthol.

Preferred actives are analgesics, antacids, decongestants, cough suppressants, expectorants, mucolytic agents and laxatives. In addition, relief agents may preferably be incorporated in the composition, either alone or in combination with other actives.

The active is preferably a solid component.

The active medicament(s) may be taste masked to further improve the taste profile of the medicinal composition. The medicament(s) may be taste masked using methods known in the art, for example adding to the core taste masking ingredients such as ethylcellulose, hydroxypropylmethylcellulose, methylethylcellulose, hydroxypropylcellulose, polyvinyl pyrrolidone, polyvinyl alcohol, mono glycerides, diglycerides, stearic acid, palmitic acid, gelatin, hydrogenated cotton seed oil and more generally any food grade polymer, starch, wax or fat. The taste masking agents may be used singly or in combination. The amount of the taste masking ingredient may be in the range by weight of the medicament(s) used.

According to a further aspect, the present invention provides a process for manufacturing the medicinal composition of the present invention.

The medicinal compositions according to the present invention may be prepared by forming a plurality of depressions (for example by vacuum forming techniques) in a first sheet of non-expanded modified cellulose material, placing the material which comprises the core into the depressions, sealing a planar second sheet of non-expanded modified cellulose material on top of the first sheet to enclose the core material, for example by adhesive or heat sealing. Preferably, also including the steps of cutting the individual dosage forms from the sheet.

Alternatively, the medicinal compositions of the present invention may be prepared by placing the core between two sheets of the non-expanded film material and sealing the sheets together around the periphery of the core. The sheets may be sealed by using an adhesive, a solvent for the material comprising the sheets, by heat or radio frequency welding. Where the core is molten, a pocket may be formed between the two sheets of material into which the molten core is placed before the open part of the pocket is sealed to enclose the molten core. After the core has been sealed between the sheets, the material may be cut either through the sealed region or around the sealed region to give the individual dosage forms which are then packed either in containers or blister packs. One example of a suitable apparatus for preparing the formulations of the present invention is described in WO-A-9735537.

The invention will now be illustrated by reference to the following examples given by way of example only.

Non-Expanded Film Preparation

The required amount of Methocel E50 (HPMC from Dow Chemical Company), plasticiser (if present), acid (if present), colourant and other components (if present) are dispersed in water and the mixture stirred vigorously for 6 hours. The resulting mixture is left to stand for 24 hours at room temperature, then a layer of the mixture is cast onto a glass sheet and the water removed by heating in an oven at 40° C.

### EXAMPLE 1

### General Preparation of Capsules

A non-expanded film of the modified cellulose material is placed over a vacuum-forming mould in which indentations of the shape of the finished dosage forms are present. The film is heated and vacuum formed to give a film with a plurality of blisters depending from a planar upper surface. Each blister is filled with the appropriate amount of core material prepared as described in Examples 2 to 31 below and a flat non-expanded film of the same modified cellulose material attached to the planar upper surface of the vacuum-formed film by applying an adhesive to both the flat film and the planar upper surface and applying pressure to ensure a good seal. The individual capsules are then separated and packed.

### EXAMPLE 2

### Capsule Containing Aqueous Fondant Core

A filled capsule was prepared as follows:

| Capsule Core | mg |
|---|---|
| Sucrose ¹ | 2100 |
| Water ² | 200 |
| Glucose syrup ² | 200 |
| Flavouring agent ⁴ | 25 |

| Capsule shell | % by weight |
|---|---|
| HPMC ⁵ | 79 |
| Polypropylene glycol | 12 |
| Glycerin | 8 |
| Colourant | 1 |

¹Celebration Sucrose NCP from British Sugar
² Distilled water
³ 42 DE Glucose Syrup from Cerestar
⁴ Cream flavour 514388E
⁵ Methocel E50 from Dow Chemical Company

The capsule of Example 2 is a placebo capsule (i.e. it contains no active agent).

The core is prepared by forming an aqueous solution by mixing the glucose syrup and water with gentle heating.(30 to 35° C.). The sucrose is then added gradually with mixing and dispersed evenly within glucose/syrup solution. Finally the flavouring agent is added thereto with mixing. The resultant capsule core is a semi-solid aqueous fondant at 25° C.

The capsule is prepared from the aqueous fondant core by the methodology of Example 1. The non-expanded capsule film has essentially no void volume and it has a thickness of about 75 μm.

### EXAMPLES 3 TO 4

### Capsules Containing Aqueous Fondant Core

A filled capsule containing the following capsule cores was prepared:

| | Ex 3 | Ex 4 |
|---|---|---|
| Core Component | mg | mg |
| Sucrose ¹ | 1615 | 2253 |
| 42 DE Glucose syrup ³ | 387 | — |
| Invert syrup No. 1 ⁶ | 350 | — |
| Gelatin | — | 37.5 |
| Water | 150 | 210 |
| Flavouring ⁴ | 25 | — |

⁶ Invert Sugar No 1 from British Sugar

The core of Example 3 was prepared in accordance with the methodology of Example 2. The core of Example 4 was prepared by dissolving the gelatin in water with heating and then mixing the sucrose into the gelatin to form a dough.

The capsule shell was identical to that used in Example 2. The non-expanded capsule film has a thickness of about 75 μm and void volume of essentially zero. The resultant capsule core is a semi-solid aqueous fondant at 25° C.

### EXAMPLE 5

### Capsule Containing Aqueous Fondant Core

A filled capsule was prepared as follows:

| Capsule Core | Mg |
|---|---|
| Sucrose ¹ | 1725 |
| Glucose ⁷ | 425 |
| Water | 350 |

⁹ Available from Cerestar

The sucrose, glucose and water are heated to 120° C. The mixture is then cooled to 45° C. and then further cooled to room temperature with mixing until a smooth and creamy texture is attained.

The capsule shell comprised the non-expanded film material identical to that used in Example 2. The non-expanded capsule film has a thickness of about 75 μm and essentially no void volume.

### EXAMPLES 6 TO 10

The following cores were produced and encapsulated in the capsule film.

| | Ex 6 | Ex 7 | Ex 8 | Ex 9 | Ex 10 |
|---|---|---|---|---|---|
| Core Component | mg | mg | mg | mg | mg |
| Sucrose ¹ | 2100 | 2100 | 1615 | 2253 | 1725 |
| 42 DE Glucose syrup ³ | 200 | 200 | 387 | — | — |
| Invert syrup No. 1 ⁶ | — | — | 350 | — | — |
| Glucose ⁷ | — | — | — | — | 425 |
| Gelatin | — | — | — | 37.5 | — |
| Water | 200 | 200 | 150 | 210 | 350 |
| Flavouring | 25 | 25 | 25 | — | — |
| Flurbiprofen | 60 | — | — | — | — |
| Taste Masked Ibuprofen | — | 225 | — | — | — |
| (90% drug) | | | | | |
| Taste masked | — | — | 350 | — | — |
| Guaiphenesin (60% drug) | | | | | |
| Dextromethorphan | — | — | — | — | 145 |
| Adsorbate (10% drug) | | | | | |
| 4-Hexylresorcinol | — | — | — | 3.2 | — |

The aqueous fondant cores of Examples 6 and 7, Example 8, Example 9 and Example 10 were prepared in accordance with the methods as detailed in Examples 2 to 5, respectively. The appropriate amount of the active medicaments is then dispersed in the aqueous fondant core with mechanical mixing. The resulting cores were filled into blisters of a non-expanded capsule film material having the following formulation.

| | % by weight |
|---|---|
| HPMC ⁵ | 79 |
| Polypropylene glycol | 12 |
| Glycerin | 8 |
| Colourant | 1 |

The capsule film was about 70 μm thick and had a void volume of essentially zero. The aqueous fondant cores of Examples 6 and 9 may be used in capsules intended for the treatment of sore throats; the aqueous fondant core of Example 7 may be used in capsules for the treatment of headaches and other similar pains or aches; the aqueous fondant core of Example 8 may be used in capsules for the treatment of chesty coughs; and the aqueous fondant core of Example 10 may be used in capsules for the treatment of dry coughs.

### EXAMPLE 11

### Aqueous Fondant Cores

Example 8 was repeated, except the sucrose component of the aqueous fondant core was replaced with fructose having a mean particle size of 15-20 μm.

### EXAMPLE 12

### Aqueous Fondant Cores

Example 9 was repeated, except the sucrose component of the aqueous fondant core was replaced with glucose having a mean particle size of 10-15 μm.

### EXAMPLES 13 AND 14

### Aqueous Fondant Cores

The following cores were produced and encapsulated in the capsule film.

| | Ex. 13 | Ex. 14 |
|---|---|---|
| Core Component | mg | mg |
| Sucrose ¹ | 2100 | 2100 |
| 42DE Glucose syrup ³ | 200 | 200 |
| Water | 200 | 200 |
| Flavouring | 25 | 25 |
| Aluminium hydroxide | 430 | 500 |
| Magnesium oxide | 70 | — |

The aqueous fondant cores were prepared in accordance with the method as detailed in Example 2. The resultant cores were filled into blisters of a non-expanded capsule film material having the following formulation.

| | % by weight |
|---|---|
| HPMC ⁵ | 75 |
| Anhydrous citric acid | 15 |
| Glycerin | 10 |

The capsule film had a thickness of about 80 μm and comprised essentially no void volume.

The aqueous fondant cores of Examples 13 and 14 are intended for the treatment of indigestion.

### EXAMPLE 15 TO 19

### Aqueous Gels

The following cores were produced for encapsulation by the capsule film:

| | Ex 15 | Ex 16 | Ex 17 | Ex 18 | Ex 19 |
|---|---|---|---|---|---|
| Core Component | mg | mg | mg | mg | mg |
| Sucrose ¹ | 385 | 1415 | 680 | — | — |
| 42DE Glucose syrup ³ | 1490 | — | — | 1915 | — |
| Carageenan | 250 | — | — | — | 250 |
| Gelatine | — | 90 | — | 50 | — |
| Sodium alginate | — | — | 100 | — | — |
| Sodium citrate | — | — | — | 12.5 | — |
| Citric acid monohydrate | — | — | — | 12.5 | — |
| Sorbistat-K | — | — | — | 7.5 | — |
| Water | 600 | 995 | 475 | 500 | 800 |
| Glycerin | — | — | — | — | 50 |
| Taste Masked Ibuprofen | — | 200 | — | — | 200 |
| (90% drug) | | | | | |
| Pseudoephedrine. HCl | 120 | 60 | — | — | — |
| Paracetamol | — | — | 500 | — | — |
| Diphenylhyramine. HCl | — | — | 25 | — | — |
| Senna | — | — | — | 7.5 | — |

The above cores are prepared in the following manner. The gelling agent and water are mixed with stirring at room temperature to produce a homogeneous mixture. The sugar (if present) and humectant (if present) are then added with mixing. The sugar may dissolve fully or partially in the gel. The one or more medicaments is then dispersed throughout the gel by mechanical mixing.

The cores of Example 15 to 19 include a thickening or viscosity modifying agent. The resultant core is an aqueous gel at 25° C. The resultant cores were filled into blisters of a non-expanded capsule film material having a void volume of essentially zero and consisting of the following formulation.

| Capsule Shell | % by weight |
|---|---|
| HPMC ⁵ | 79 |
| Propylene glycol | 12 |
| Glycerin | 8 |
| Colourant | 1 |

The capsule film had a thickness of about 80 μm and essentially zero void volume.

The core of Examples 15, 16, 17 and 19 may be used in capsules for treating cold and flu symptoms and the core of Example 18 may be used in capsules for treating indigestion.

### EXAMPLE 20 TO 24

### Thickened Oils

The following cores were produced and encapsulated by the capsule film as used in Examples 13 to 16.

| | Ex 20 | Ex 21 | Ex 22 | Ex 23 | Ex 24 |
|---|---|---|---|---|---|
| Core Component | mg | mg | mg | mg | mg |
| Olive Oil ⁸ | 1600 | 1500 | — | — | — |
| Geloil SC ⁹ | — | — | 1574 | 1500 | 1500 |
| Glyceryl monostearate | 400 | 350 | — | — | — |
| Aerosil 200 ¹⁰ | — | — | 94 | 90 | 90 |
| Sucrose ¹ | — | — | 316 | 300 | 300 |
| Flavour ¹¹ | — | — | 16 | 10 | 10 |
| Taste Masked Ibuprofen | 250 | — | 230 | — | — |
| (90% drug content) | | | | | |
| Pseudoephedrine HCl | — | — | 60 | — | — |
| Guiaphenesin (60% drug) | — | 300 | — | — | — |
| Aluminium hydroxide | — | — | — | 500 | — |
| Paracetamol | — | — | — | — | 400 |

⁸ Available from Olio Carli, Imperia
⁹ Geloil SC is available from Gattetasse and comprises soyabean oil, C₁₆₋₁₈ mono-, di and tri-glycerides, and polyglyceryl oleate.
¹⁰ Aerosil 200 is colloidal silica available from Degussa
¹¹ Blackcurrant flavour 17.80.3606

The cores of Examples 20 to 22 are prepared by heating the oil component to approximately 70° C. and then the thickening agent, sugar (if present) and flavouring (if present) added thereto with stirring. The mixture is then cooled to room temperature to provide a gel. The active medicament(s) is/are dispersed within the cooled gel. Examples 23 and 24 are prepared in the same manner except no heating is required.

The non-expanded capsule film had a thickness of about 80 μm and essentially zero void volume.

### EXAMPLE 25

### Thickened Oil-in-Water Emulsion

The following core was produced and encapsulated by the film as used in Examples 15 to 19.

| Core Component | mg |
|---|---|
| 42DE Glucose syrup ³ | 1050 |
| Hydrogenated coconut oil ¹² | 525 |
| Gelatine | 25 |
| Water | 125 |
| Sucrose ¹ | 350 |
| Taste Masked Ibuprofen | 250 |
| (90% drug content) | |

¹² Kristal Special - available from Karlshamns

The gelatine is dissolved in water with heating and then mixed with the glucose syrup to form the aqueous phase. The sucrose is added to the aqueous phase with mixing. The hydrogenated coconut oil is heated and the two phases combined with high shear mixing. The medicament is then added to the emulsion with mixing.

The film had a thickness of about 80 μm and essentially zero void volume.

### EXAMPLES 26 TO 28

### Thickened Oil-in-Water Emulsion

The following cores were produced by the method as outlined in Example 25 and encapsulated within the non-expanded film as used in Example 2.

| | Ex 26 | Ex 27 | Ex 28 |
|---|---|---|---|
| Core Component | mg | mg | mg |
| Olive Oil ⁸ | — | 450 | 450 |
| Geloil SC ⁹ | 400 | — | — |
| Gelatine | 30 | 30 | 30 |
| 42DE Glucose syrup ³ | 1050 | 1050 | 1050 |
| Sucrose ¹ | 350 | 350 | 350 |
| Pseudophedrine HCl | 50 | — | — |
| Paracetamol | — | 500 | — |
| Taste Masked Ibuprofen | — | — | 200 |
| (90% drug content) | | | |

### EXAMPLES 29 AND 30

### Thickened Water-in-Oil Emulsions

The following cores were produced and encapsulated by the non-expanded film as used in Example 2.

| | Ex 20 | Ex 21 |
|---|---|---|
| Core Component | mg | mg |
| Miglyol 812 ¹³ | 200 | — |
| 42DE Glucose syrup ³ | 50 | 28 |
| Soya lecithin | 20 | — |
| Hydrogenated coconut oil ¹² | — | 75 |
| Taste masked Ibuprofen | 200 | — |
| (90% drug content) | | |
| Pseudoephidrine-HCl | — | 70 |

¹³ Miglyol 812 available from Hüls

The Miglyol was mixed with the soya lecithin at room temperature and then combined with the glucose syrup aqueous phase with vigorous stirring. The medicament was then dispersed in the emulsion by mechanical mixing. Example 30 was prepared in the same manner, except the hydrogenated coconut oil was heated prior to mixing with the soya lecithin.

### EXAMPLE 31

### Non Aqueous Emulsion

The following core was produced and encapsulated by the non-expanded film as used in Example 2. The film capsule has a thickness of about 75 μm and essentially zero void volume.

| Core Component | mg |
|---|---|
| Glycerin | 1570 |
| Miglyol 812 ¹³ | 750 |
| Cremophor RH40 ¹⁴ | 112 |
| Taste masked Ibuprofen (90% drug content) | 225 |

¹⁴ Cremophor RH40 is a polyoxyethylene caster oil derivative available from BASF

The Cremophor and glycerin are combined with stirring and then mixed with Miglyol 812. The medicament is dispersed in the resultant emulsion with mixing.

1. A medicinal composition comprising:
(a) a core in combination with a medicinally effective unit dose of one or more active medicaments;
(b) the core being enclosed within a non-expanded film material comprising a modified cellulose material.

2. A medicinal composition as claimed in claim 1 wherein the non-expanded film material has a void volume which represents less than or equal to 15% by volume of the total volume of the non-expanded film material.

3. A medicinal composition as claimed in claim 1 wherein the non expanded film material includes a first major surface having an essentially smooth surface texture extending across a part of or the entire surface.

4. A medicinal composition as claimed in claim 1 go wherein the non-expanded film material includes a first major surface having a roughness average of less than or equal to 3,μm (as measured in accordance with ASME B46-1-1995) extending across a part of or the entire surface.

5. A medicinal composition as claimed in claim 1 wherein the core has a viscosity of greater than or equal to 100 Pals. when measured at 25° C. at a shear stress of 1 Pa.

6. A medicinal composition as claimed in claim 1 so wherein the core exhibits a peak normal force of greater than or equal to 0.1 N during a squeeze flow test conducted at 25° C. over 500 seconds at a compression rate of 10, μm/sec.

7. A medicinal composition as claimed in claim 1 wherein the core is selected from an aqueous fondant core, a thickened oil, an emulsion, or an aqueous gel.

8. A medicinal composition as claimed in claim 7, wherein the core is an aqueous fondant core which comprises greater than or equal to 50% by weight of one or more sugars and less than or equal to 50% by weight of an aqueous carrier, especially water.

9. A medicinal composition as claimed in claim 8, wherein the core further includes 0.5 to 20% by weight of one or more thickening or viscosity modifying agents.

10. A medicinal composition as claimed in claim 7, wherein the core is a s thickened oil which comprises greater than or equal to 50% by weight of one or more oils and less than or equal to 50% by weight of one or more thickening or viscosity modifying agents.

11. A medicinal composition as claimed in claim 10 wherein the one or more go oils includes 20 to 30% by weight, based on the total weight of the one or more oil, of one or more sugars.

12. A medicinal composition as claimed in claim 7, wherein the core is an aqueous gel comprising greater than or equal to 70% by weight of an s aqueous phase and less than or equal to 30% by weight of one or more thickening or viscosity modifying agents.

13. A medicinal composition as claimed in claim 7, wherein the core is selected from an oil-in-water type emulsion, a water-in-oil type emulsion, or a non aqueous emulsion.

14. A medicinal composition as claimed in claim 1 wherein the non-expanded film material comprises at least 40% by weight of the cellulose material.

15. A medicinal composition as claimed in claim 1 wherein the cellulose material of the non-expanded film material comprises hydroxypropyl methyl cellulose.

16. A medicinal composition as claimed in claim 1 wherein the non-expanded film material consists essentially of 40-80% by weight hydroxypropyl methyl cellulose and 20-60% by weight of one or more plasticizers.

17. A medicinal composition according to claim 16, wherein the one or more plasticisers is selected from polyethyleneglycols, diacetin, propylene glycols and glycerin.

18. A medicinal composition as claimed in claim 1 wherein the non-expanded film material has a thickness of less than or equal to 100 μm.
